(19) United States
(12) Patent Application Publication
(10) Pub. No.: US 2006/0258784 A1
(43) Pub. Date: Nov. 16, 2006

(54) New compounds, a process for their preparation and their use as dyes and pigments

(76) Inventors: Jean-Marie Adam, Rosenau (FR); Pramod V. Dalvi, Atul (IN); Vadiraj Subbanna Ekkundi, Aesch (CH); Jean-Pierre Bacher, Buschwiller (FR); Ramaswami Sreenivasan, Mumbai (IN); Deepak M. Rane, Mumbai (IN)

Correspondence Address: Ciba Specialty Chemicals Corporation;Patent Department, 540 White Plains Road, P.O. Box 2005, Tarrytown, NY, 10591-9005, US

(21) Appl. No.: 11/487,840

(22) Filed: Jul. 17, 2006

Related U.S. Application Data
(62) Division of application No. 10/548,358, filed on Jan. 12, 2006.

(30) Foreign Application Priority Data
Mar. 19, 2003 (IN) 228/MAS/2003

(51) Int. Cl. C07D 413/04; C08K 5/34

(52) U.S. Cl. 524/90; 548/228

(57) ABSTRACT

Compounds of formula (1) 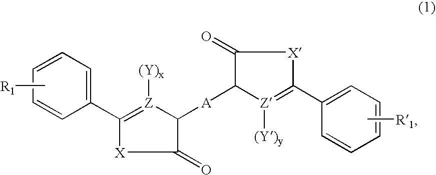 wherein
R₁ is hydrogen, hydroxy, halogen, nitro, cyano, amino, carboxy, carboxylic ester, sulfo, sulfonic ester, carboxylic amide, sulfonic amide, alkylthio, arylthio, alkoxy or aryloxy, R′₁ is hydrogen, hydroxy, halogen, nitro, cyano, amino, carboxy, carboxylic ester, sulfo, sulfonic ester, carboxylic amide, sulfonic amide, alkylthio, arylthio, alkoxy or aryloxy, X is —O—, —S—, —NH— or —N(alkyl)—, X′ is —O—, —S—, —NH— or —N(alkyl)—, Y is hydrogen or carboxylic ester, Y′ is hydrogen or carboxylic ester, Z is ═C— or ═N—, Z′ is ═C— or ═N—, x is 0 or 1, when Z is ═N—, then x is 0, y is 0 or 1, when Z′ is ═N—, then y is 0, A is a conjugated linking bridge of the formula 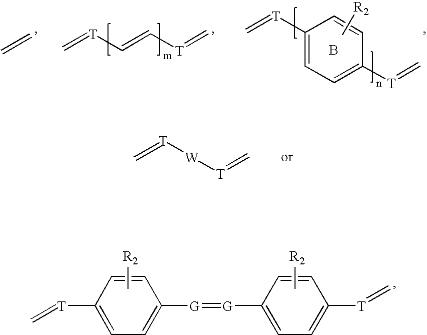 wherein
n is 0, 1, 2 or 3, m is 0, 1, 2 or 3, B is a phenyl ring, T is ═C(R₃)— or ═N—, wherein R₃ is hydrogen, C₁-C₁₂alkyl or CN, W is a heterocyclic, or linear or polycondensed aromatic group which is unsubstituted or substituted by alkyl, halogen, hydroxy, alkoxy, alkylthio or amino, G is —CH═ or —N═, and R₂ is hydrogen, alkyl, halogen, hydroxy, alkoxy, alkylthio or amino, their preparation and their use in the production of coloured plastics or polymeric colour particles.

This is a divisional of U.S. application Ser. No. 10/548,358 pending, which is a 371 of PCT/EP 04/050276 filed Mar. 10, 2004, which application is hereby incorporated by reference.

The present invention relates to new compounds, to their preparation and to their use in the production of coloured plastics or polymeric colour particles.

The subject of the present invention are compounds of formula (1) 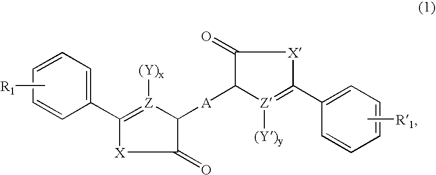
wherein
R₁ is hydrogen, hydroxy, halogen, nitro, cyano, amino, carboxy, carboxylic ester, sulfo, sulfonic ester, carboxylic amide, sulfonic amide, alkylthio, arylthio, alkoxy or aryloxy,
R′₁ is hydrogen, hydroxy, halogen, nitro, cyano, amino, carboxy, carboxylic ester, sulfo, sulfonic ester, carboxylic amide, sulfonic amide, alkylthio, arylthio, alkoxy or aryloxy,
X is —O—, —S—, —NH— or —N(alkyl)-,
X′ is —O—, —S—, —NH— or —N(alkyl)-,
Y is hydrogen or carboxylic ester,
Y′ is hydrogen or carboxylic ester,
Z is ═C— or ═N—,
Z′ is ═C— or ═N—,
x is 0 or 1, when Z is ═N—, then x is 0,
y is 0 or 1, when Z′ is ═N—, then y is 0,
A is a conjugated linking bridge of the formula 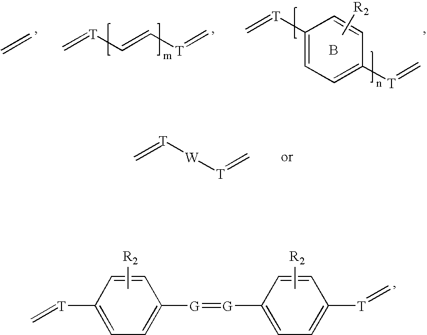
   wherein
n is 0, 1, 2 or 3,
m is 0, 1, 2 or 3,
B is a phenyl ring,
T is ═C(R₃)— or ═N—, wherein R₃ is hydrogen, C₁-C₁₂alkyl or CN,
W is a heterocyclic, or linear or polycondensed aromatic group which is unsubstituted or substituted by alkyl, halogen, hydroxy, alkoxy, alkylthio or amino,
G is —CH═ or —N═, and
R₂ is hydrogen, alkyl, halogen, hydroxy, alkoxy, alkylthio or amino.

According to the invention an alkyl is for example a straight-chain or branched C₁₋₈alkyl as methyl, ethyl, n-propyl, isopropyl, n-butyl, sec-butyl, isobutyl, tert-butyl, n-pentyl, 2-pentyl, 3-pentyl, 2,2-dimethylpropyl, hexyl, heptyl, 2,4,4-trimethylpentyl, 2-ethylhexyl or octyl, preference being given to a C₁₋₄alkyl.

According to the invention an alkylthio is for example methylthio, ethylthio, propylthio, butylthio, heptylthio or hexylthio.

According to the invention an alkoxy is for example a straight-chain or branched C₁₋₈alkoxy, for example methoxy, ethoxy, n-propoxy, isopropoxy, n-butoxy, sec-butoxy, isobutoxy, tert. butoxy, n-pentyloxy, 2-pentyloxy, 3-pentyloxy, 2,2-dimethylpropoxy, n-hexyloxy, n-heptyloxy, n-octyloxy, 1,1,3,3-tetramethylbutoxy or 2-ethylhexyloxy.

According to the invention an aryloxy is to be understood as being for example a C₆₋₂₄aryloxy, preferably a C₆₋₁₂aryloxy radical, for example phenoxy or 4-methylphenoxy.

According to the invention an arylthio is for example phenylthio or napthylthio.
G is preferred —CH═.
W as aromatic group includes, for example, phenylene, naphthalene, acenaphthylene, anthracene, phenanthrene, naphthacene, chrysene, pyrene or perylene. W is preferably phenylene, naphthalene, anthracene, phenanthrene, perylene or pyrene, and most preferably phenylene or naphthalene.
W as heterocyclic group is, for instance, pyridine, pyrazine, pyrimidine, pyridazine, indole isoindole, quinoline, isoquinoline, carbazole, phenothiazine, benzimidazolone, benzothiazole, pyrrolo, imidazole, pyrrolidine, piperidine, piperazine, morpholine or pyrazole. According to the invention an ester is for example methyl-, ethyl-, propyl- or butylester.

If A is a single double bond ═, preferred compounds of formula (1) are
a) compounds of phenyl-butyrolactams of formula 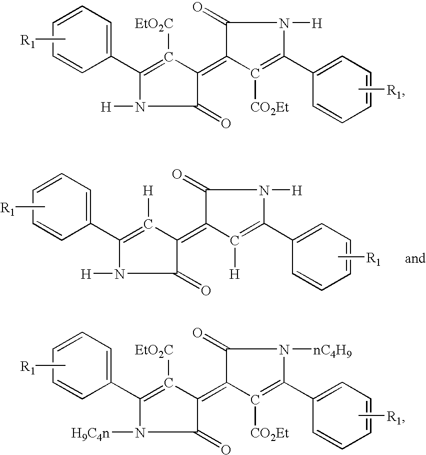
b) compounds of phenyl-butyrolactones of formula 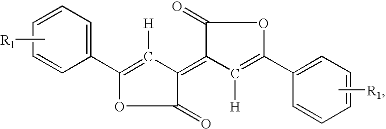
c) compounds of phenyl oxazolones of formula 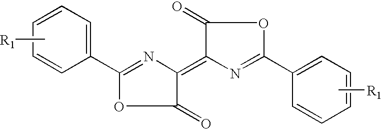
   and
d) compounds of phenyl imidazolones of formula 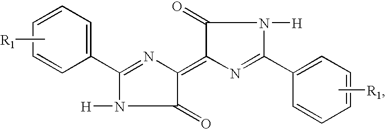
   wherein R₁ has the meaning given under the formula (1) and Et is —CH₂CH₃.

If A is a conjugated linking bridge, preferred compounds of formula (1) are
e) compounds of phenyl-carboethoxy-butyrolactams of formula 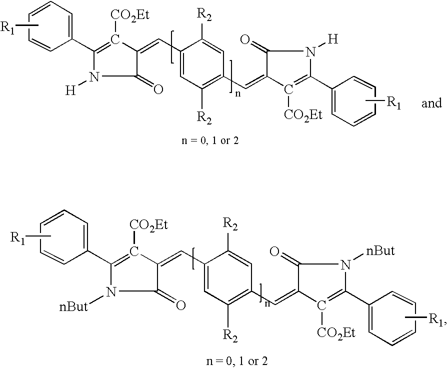
f) compounds of phenyl-butyrolactones of formula 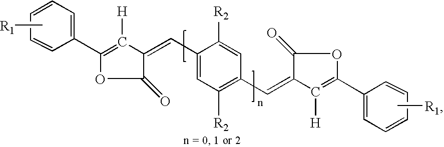
§g) compounds of phenyl-butyrolactams of formula 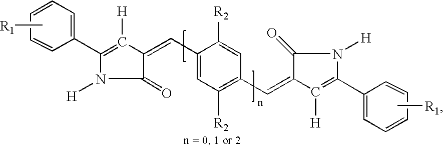
h) compounds of phenyl-oxazolones of formula 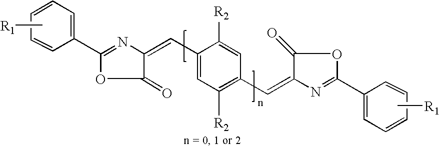
   and
i) compounds of phenyl-imidazolones of formula 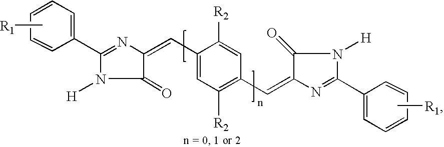
   wherein R₁ and R₂ have the meaning given under the formula (1), Et is —CH₂CH₃ and nBut is n-butanole.

Further preferred are compounds of formula (1), wherein R₁ is hydrogen, chloro, bromo, methyl, methoxy, ethoxy, tert.-butyl, phenyl or nitro is, R′₁ is hydrogen, chloro, bromo, methyl, methoxy, ethoxy, tert.-butyl, phenyl or nitro, R₂ is hydrogen or methoxy, X is —NH—, —N(n-C₄H₉)—, or —O—, X′ is —NH—, —N(n-C₄H₉)—, or —O—, Y is hydrogen or COOC₂H₅, Y═ is hydrogen or COOC₂H₅, Z is ═C— or ═N—, Z′ is ═C— or ═N—, A is ═ or 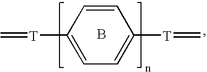
T is ═C(R₃)—, R₃ is hydrogen and n is 0, 1 or 2.

The most preferred compounds of formula (1) are the compounds of the formulae

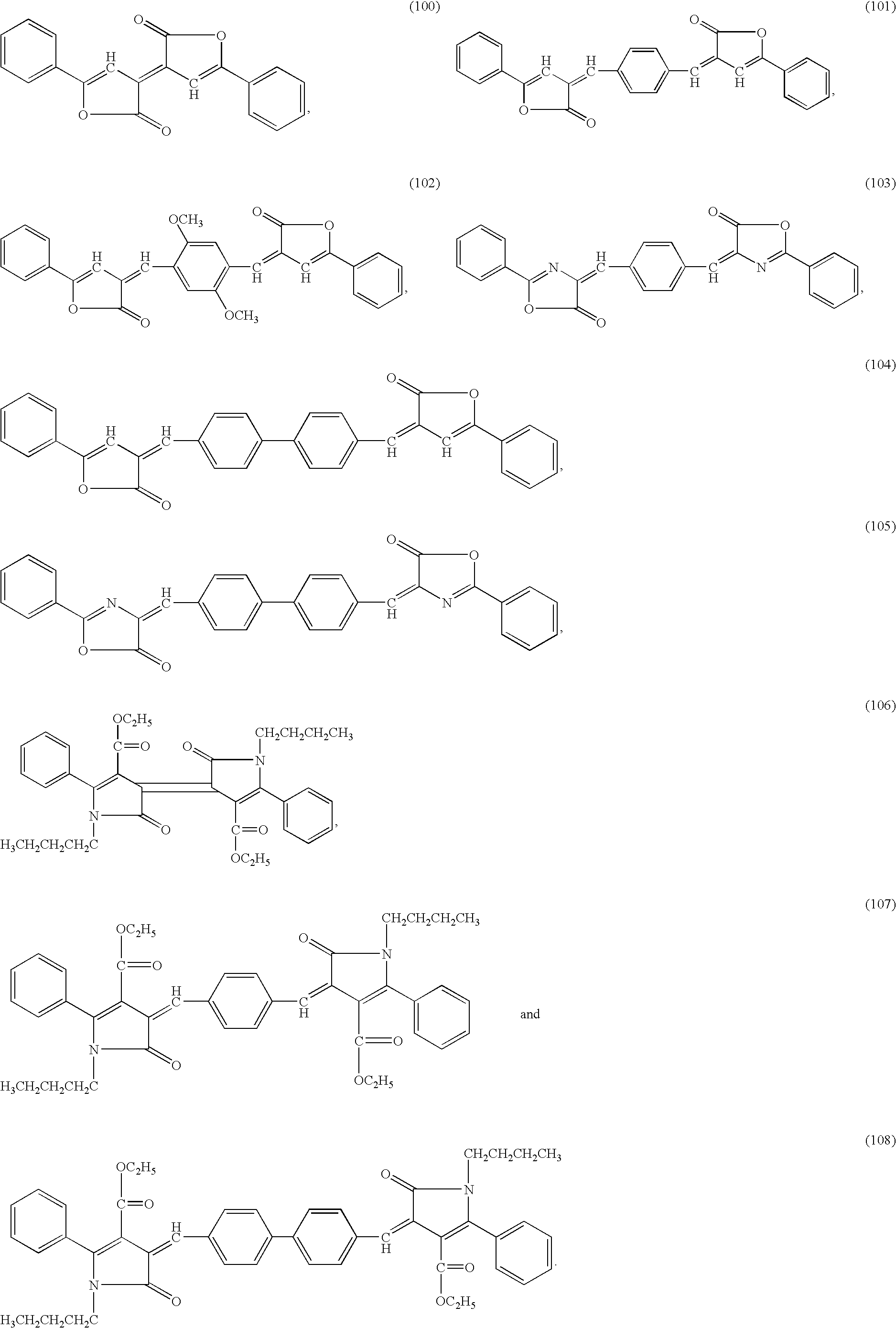

The compounds of formula (1) according to the invention are prepared, for example, by reacting 2 mol of compound of formula 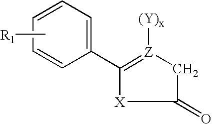
(50) which possess an active methylene group —CH₂ or 1 mol of compound of formula (50) and 1 mol of compound of formula 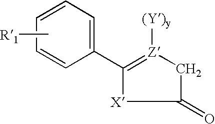
(51), which possess an active methylene group —CH₂ with 1 mol of one of the compounds of formula 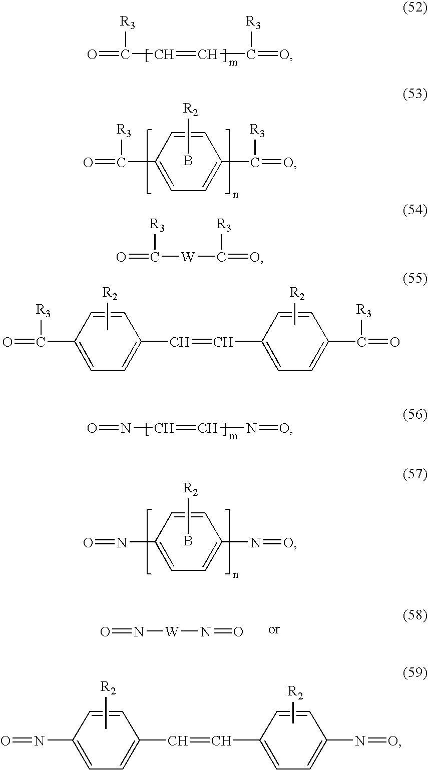
wherein R₁, R′₁, X, X′, Y, Y′, Z, Z′, x and y are as defined above for formula (1), n is 0, 1, 2 or 3, m is 0, 1, 2 or 3, B is a phenyl ring, R₂ is hydrogen, alkyl, halogen, hydroxy, alkoxy, alkylthio or amino and R₃ is hydrogen, C₁-C₁₂alkyl or CN, at elevated temperature
or
by oxidation 2 mol of compound of formula (50) at elevated temperature
or
by oxidation 1 mol of compound of formula (50) and 1 mol of compound of formula (51) at elevated temperature.

The general synthesis of phenyl-butyrolactams (in this case C-substituted by carboethoxy) derivatives is characterized by the following reaction schema: 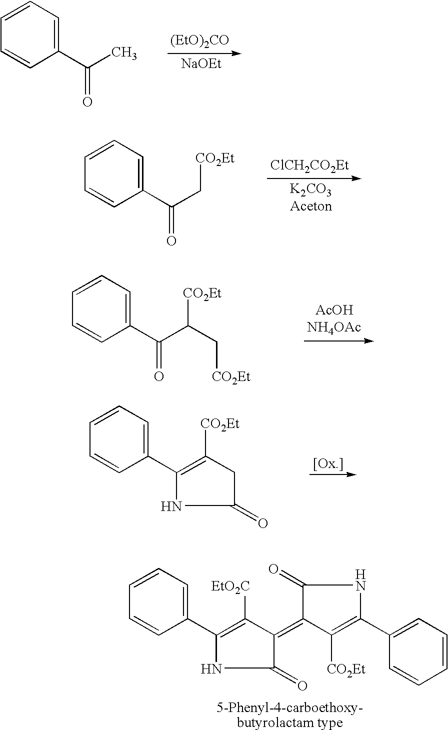
wherein Ac means acetic acid residue and Et means ethyl.

The general synthesis of phenyl-butyrolactam and phenyl-butyrolactone (N- and C- unsubstituted) derivatives is characterized by the following reaction schema: 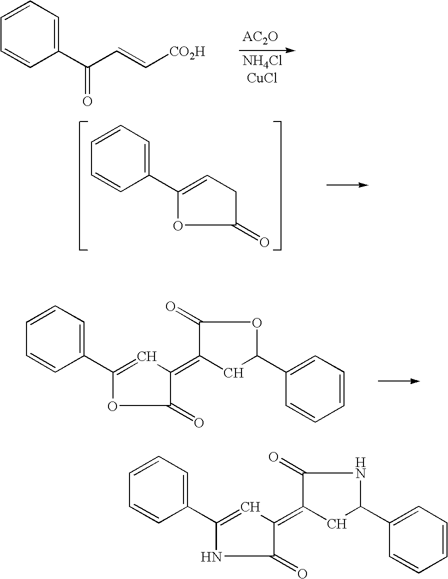
wherein Ac₂O means acetic anhydride.

The general synthesis of phenyl-oxazolone derivatives is characterized by the following reaction schema: 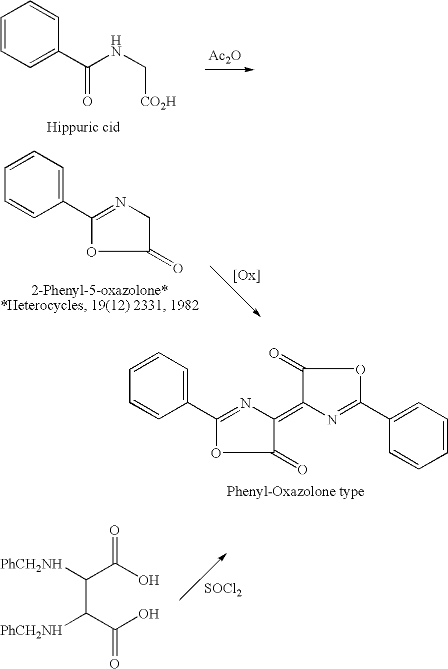
wherein Ph means phenyl and Ac₂O means acetic anhydride

The general synthesis of pheny-imidazolone derivatives is characterized by the following reaction schema: 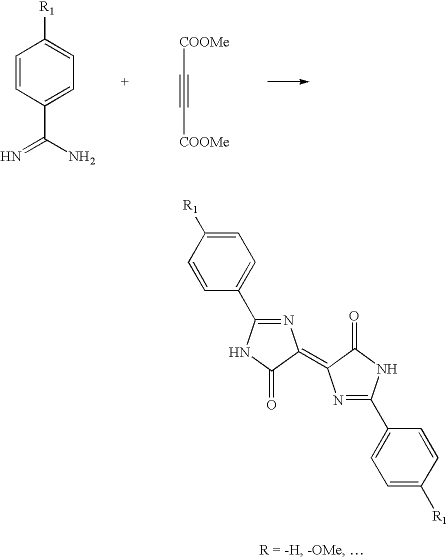
wherein R₁ has the meaning given under the formula (1) and Me is methyl.

The general synthesis based on the condensation of a dialdehyde with an active methylene compound is characterized by the following reaction schema: 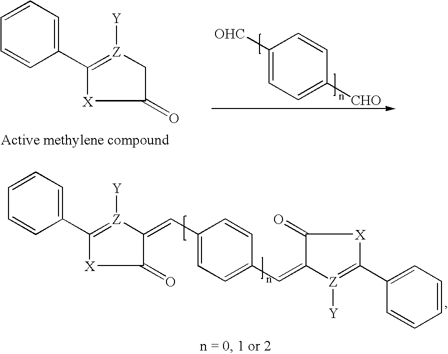
wherein X, Y and Z have the meaning given under the formula (1).

The compound of formula (1) can be symmetrical or asymmetrical and can contain one or more water soluble groups (sulfonic, carboxylic or cationic groups).

Water soluble derivatives of compounds of formula (1) can be used as dyestuffs for textile application, coloration of cotton, wool, polyamide and polyacrylonitrile using all the well known dyeing processes.

Such dyes are useful for dyeing and printing manufactured natural polymer and especially synthetic hydrophobic fibre materials, especially textile materials. Textile materials composed of blend fabrics comprising such manufactured natural polymer or synthetic hydrophobic textile materials are likewise dyeable or printable with the dyes of the invention.

Useful manufactured natural polymer textile materials are especially cellulose acetate and cellulose triacetate.

Synthetic hydrophobic textile materials are especially linear aromatic polyesters, for example polyesters formed from a terephthalic acid and glycols, particularly ethylene glycol, or condensation products of terephthalic acid and 1,4-bis(hydroxymethyl)cyclohexane; polycarbonates, for example those formed from α,α-dimethyl-4,4-dihydroxydiphenylmethane and phosgene; or fibres based on polyvinyl chloride or polyamide.

The above dyes are applied to the textile materials according to known dyeing processes. For example, polyester fibres are exhaust dyed from an aqueous dispersion in the presence of customary anionic or nonionic dispersants with or without customary carriers at temperatures between 80 and 140° C. Cellulose acetate is preferably dyed at between about 65 to 85° C. and cellulose triacetate at up to 115° C.

The above dyes are also useful for dyeing by the thermosol, exhaust and continuous processes and for printing processes. The exhaust process is preferred. The liquor ratio depends on the apparatus, the substrate and the make-up form. However, the liquor ratio can be chosen to be within a wide range, for example in the range from 4:1 to 100:1, but it preferably is between 6:1 to 25:1.

The textile material mentioned may be present in the various processing forms, for example as a fibre, yarn or web or as a woven or loop-formingly knitted fabric.

It is advantageous to convert the dyes into a dye preparation before use. For this, the dyes are ground so that their particle size is on average between 0.1 and 10 microns. The grinding may be effected in the presence of dispersants. For example, the dried dye is ground with a dispersant or kneaded in paste form with a dispersant and then dried under reduced pressure or by spray drying. The preparations thus obtained can be used to prepare print pastes and dyebaths by adding water.

Printing utilizes the customary thickeners, for example modified or nonmodified natural products, for example alginates, British gum, gum arabic, crystal gum, carob bean flour, tragacanth, carboxymethylcellulose, hydroxyethylcellulose, starch or synthetic products, for example polyacrylamides, polyacrylic acid or copolymers thereof or polyvinyl alcohols.

The above dyes confer on the materials mentioned, especially on polyester material, level shades having very good service fastnesses, such as in particular good light fastness, especially a very good hot light fastness, fastness to dry heat setting and pleating, chlorine fastness and wet fastness such as fastness to water, perspiration and washing; the dyeings are further characterized by good rub fastness and heat stability.

Water insoluble derivatives of compounds of formula (1) can be used as disperse dyes for coloration of PET by exhaustion or pigments for mass coloration of plastics or can be used for inks and paints. These products can also be used for coloration of wood and metals and they are also suitable as functional dyes for special applications such as optical information storage, or display devices or printed circuit boards.

The present invention relates also to a process for the production of coloured plastics or polymeric colour particles, which comprises mixing together a high molecular weight organic material and a tinctorially effective amount of at least one compound of formula (1).

The present invention further relates to the use of the compounds of formula (1) individually as colourants, especially for colouring or pigmenting organic or inorganic, high-molecular-weight or low-molecular-weight material, especially high-molecular-weight organic material. It is also possible, however, for the compositions according to the invention comprising compounds of formula (1) to be used in the form of mixtures, solid solutions or mixed crystals. Compounds of formula (1) can also be combined with colourants of another chemical class, for example with dyes or pigments, for example those selected from the group of the diketopyrrolopyrroles, quinacridones, perylenes, dioxazines, anthraquinones, indanthrones, flavanthrones, indigos, thioindigos, quinophthalones, isoindolinones, isoindolines, phthalocyanines, metal complexes, azo pigments and azo dyes.

The high-molecular-weight material may be organic or inorganic and may be synthetic and/or natural material. The high-molecular-weight organic material usually has an average molecular weight of 10⁵-10⁷ g/mol. It may be, for example, a natural resin or a drying oil, rubber or casein or a modified natural material, such as chlorinated rubber, oil-modified alkyd resins, viscose, or a cellulose ether or ester, such as ethylcellulose, cellulose acetate, propionate or butyrate, cellulose acetobutyrate or nitrocellulose, but is especially a completely synthetic organic polymer (duroplasts and thermoplasts) as may be obtained by polymerisation, for example by polycondensation or polyaddition. The class of polymers includes, for example, polyolefins, such as polyethylene, polypropylene, polyisobutylene, and substituted polyolefins, such as polymerisates of monomers such as vinyl chloride, vinyl acetate, styrene, acrylonitrile, acrylates, methacrylates, fluoropolymers, such as polyfluoroethylene, polytrifluorochloroethylene or tetrafluoroethylene/hexafluoropropylene mixed polymerisate, and copolymerisates of the mentioned monomers, especially ABS (acrylonitrile/butadiene/styrene) or EVA (ethylene/vinyl acetate). From the group of polyaddition and polycondensation resins it is possible to use, for example, condensation products of formaldehyde with phenols, the so-called phenoplasts, and condensation products of formaldehyde and urea or thiourea, also melamine, the so-called aminoplasts, and the polyesters used as surface coating resins, either saturated, such as alkyd resins, or unsaturated, such as maleic resins, and also linear polyesters, polyamides, polyurethanes, polycarbonates, polyphenylene oxides or silicones, and silicone resins. The mentioned high-molecular-weight compounds may be present individually or in mixtures in the form of kneadable compounds, melts or in the form of spinning solutions. They may also be present in the form of their monomers or in the polymerised state in dissolved form as film-formers or binders for paints or printing inks, such as, for example, boiled linseed oil, nitrocellulose, alkyd resins, melamine resins and urea-formaldehyde resins or acrylic resins.

Low-molecular-weight materials are, for example, mineral oils, waxes or lubricating greases.

The present invention further relates, therefore, to the use of the compounds of formula (1) for the production of inks, for printing inks in printing processes, for flexographic printing, screen printing, the printing of packaging, security colour printing, intaglio printing or offset printing, for preliminary printing stages and for textile printing, for office and home use or for graphics, such as, for example, for paper goods, for ball-point pens, felt-tip pens, fibre-tip pens, paperboard, wood, (wood) stains, metal, stamp pads or inks for impact printing processes (with impact printing ink ribbons), for the production of colourants, for paints, for use in industry or advertising, for textile decoration and industrial labelling, for roll coating or powder coating compositions or for automobile paints, for high-solids (low-solvent), water-containing or metallic paints or for pigmented formulations for aqueous paints, for mineral oils, lubricating greases or waxes, for the production of coloured plastics for coatings, fibres, plates or moulded substrates, for the production of non-impact printing material for digital printing, for the thermal wax-transfer printing process, the ink-jet printing process or for the thermal transfer printing process, and also for the production of colour filters, especially for visible light in the range of from 400 to 700 nm, for liquid crystal displays (LCDs) or charge-coupled devices (CCDS) or for the production of cosmetics or for the production of polymeric colour particles, toners, dry copy toners, liquid copy toners or electrophotographic toners.

The present invention further relates to inks comprising high-molecular-weight organic material and a colour-producing amount of the compound of formula (1).

For example, the inks can be produced by mixing the compounds according to the invention with polymeric dispersants.

The mixing of the compounds according to the invention with the polymeric dispersant is preferably carried out by generally known mixing methods, such as stirring or mixing, and the use of an intensive mixer, such as an Ultraturax, is especially to be recommended.

When mixing the compounds according to the invention with polymeric dispersants, a water-dilutable organic solvent is advantageously used.

The weight ratio of the compounds according to the invention to ink is advantageously selected to be in the range of from 0.0001 to 75% by weight, preferably from 0.001 to 50% by weight, based on the total weight of the ink.

The present invention therefore relates also to a process for the production of inks which comprises mixing high-molecular-weight organic material with a colour-producing amount of the compound of formula (1).

The present invention further relates to colourants comprising high-molecular-weight organic material and a compound according to the invention of formula (1) in a colour-producing amount.

The present invention relates, in addition, to a process for the preparation of colourants which comprises mixing a high-molecular-weight organic material and a colour-producing amount of the compound according to the invention of formula (1).

The present invention further relates to coloured or pigmented plastics or polymeric coloured particles comprising high-molecular-weight organic material and compound of formula (1) in a colour-producing amount.

The present invention relates, in addition, to a process for the preparation of coloured or pigmented plastics or polymeric coloured particles which comprises mixing together a high-molecular-weight organic material and a colour-producing amount of the compound of formula (1).

The colouring of high-molecular-weight organic substances with the colourants of formula (1) is carried out, for example, by mixing such a colourant, optionally in the form of a master batch, into those substrates using roll mills or mixing or grinding apparatus, whereby the colourant is dissolved or finely distributed in the high-molecular-weight material. The high-molecular-weight organic material with the admixed colourant is then processed according to procedures known per se, such as, for example, calendering, compression moulding, extrusion moulding, coating, spinning, casting or injection-moulding, whereby the coloured material acquires its final form. Admixing of the colourant can also be carried out immediately prior to the actual processing step, for example by continuously metering a powdered colourant according to the invention and a granulated high-molecular-weight organic material, and optionally also additional ingredients, such as additives, directly into the inlet zone of an extruder simultaneously, where mixing takes place just before the processing operation. In general, however, prior mixing of the colourant into the high-molecular-weight organic material is preferred, since more uniform results can be obtained.

It is often desirable for the purpose of producing non-rigid mouldings or reducing the brittleness thereof to incorporate so-called plasticisers into the high-molecular-weight compounds before shaping. There may be used as plasticisers, for example, esters of phosphoric acid, phthalic acid or sebacic acid. In the process according to the invention, the plasticisers can be incorporated into the polymers before or after the incorporation of the colourant. In order to obtain different colour shades it is also possible to add to the high-molecular-weight organic substances, in addition to the compounds of formula (1), any desired amounts of constituents such as white, coloured or black pigments.

For the colouring of paints and printing inks, the high-molecular-weight organic materials and the compounds of formula (1) optionally together with additional ingredients, such as fillers, dyes, pigments, siccatives or plasticisers, are finely dispersed or dissolved in a common organic solvent or solvent mixture. That procedure may comprise dispersing or dissolving each individual component on its own or dispersing or dissolving several components together and only then combining all the components. Processing is carried out in accordance with customary methods, for example by spraying, film-spreading or one of the many printing methods, whereupon the paint or printing ink is advantageously cured thermally or by irradiation, optionally after previous drying.

When the high-molecular-weight material to be coloured is a paint, it may be a conventional paint or a special paint, for example an automobile finish, preferably a metal-effect finish containing, for example, metal or mica particles.

Preference is given to the colouring of thermoplastics, especially also in the form of fibres, and printing inks. Preferred high-molecular-weight organic materials that can be coloured according to the invention are, very generally, polymers having a dielectric constant ≧2.5, especially polyesters, polycarbonate (PC), polystyrene (PS), polymethylmethacrylate (PMMA), polyamide, polyethylene, polypropylene, styrene/acrylonitrile (SAN) or acrylonitrile/butadiene/styrene (ABS). More especially preferred are polyesters, polycarbonate, polystyrene and PMMA. Most especially preferred are polyesters, polycarbonate and PMMA, especially aromatic polyesters that can be obtained by polycondensation of terephthalic acid, such as, for example, polyethylene terephthalate (PET) or polybutylene terephthalate.

They can be used in the form of their monomers or copolymers or in the polymerised state in dissolved form as film formers or binders for paints that can be used for the decoration of metal or for decorative colour finishes, and for printing inks used, for example, in the ink-jet printing process, or also for wood stains.

Special preference is also given to the colouring of mineral oils, lubricating greases and waxes with the compounds according to the invention.

The present invention also relates to mineral oils, lubricating greases and waxes comprising high-molecular-weight organic material and a compound of formula (1), in a colour-producing amount.

The present invention also relates to a process for the preparation of mineral oils, lubricating greases and waxes, which comprises mixing high-molecular-weight organic material with a colour-producing amount of the compound of formula (1).

The present invention also relates to non-impact printing material comprising high-molecular-weight organic material and a compound of formula (1), in a colour-producing amount.

The present invention relates, in addition, to a process for the preparation of non-impact printing material, which comprises mixing together a high-molecular-weight organic material and a colour-producing amount of the compound of formula (1).

The present invention further relates to a process for the production of colour filters comprising a transparent substrate and a red, a blue and a green coating applied thereto in any desired sequence, which comprises using for the production of the red, blue and green coatings a correspondingly coloured compound of formula (1).

The different-coloured coatings are preferably arranged in such a pattern that they do not overlap over at least 5% of their respective surface area and, most preferably, do not overlap at all.

The colour filters can be coated, for example, using inks, especially printing inks, comprising the compounds according to the invention, or, for example, by mixing a compound according to the invention with a chemically, thermally or photolytically structurable high-molecular-weight material (resist). The further production can be carried out, for example, analogously to EP-A-654 711, by application to a substrate, such as an LCD, subsequent photo-structuring and developing.

The invention further includes a transparent substrate coated with a red, a blue and a green coating each of a correspondingly coloured compound of formula (1), comprising pigmented high-molecular-weight organic material.

The sequence in which coating is carried out is not important as a rule. The different-coloured coatings are preferably arranged in such a pattern that they do not overlap over at least 5% of their respective surface area and, most preferably, do not overlap at all.

The present invention also includes colour filters comprising a transparent substrate and, applied thereto, a red, a blue and a green coating, each obtainable from a correspondingly coloured compound of formula (1).

The present invention also includes the use of the compounds of formula (1) for optical information storage applications (ois).

The present invention relates, in addition, to toners comprising high-molecular-weight organic material and a compound of formula (1), in a colour-producing amount.

The present invention also relates to a process for the production of toners, which comprises mixing together a high-molecular-weight organic material and a colour-producing amount of the compound of formula (1).

The present invention also relates to inks or colourants for paints, printing inks, mineral oils, lubricating greases or waxes, or coloured or pigmented plastics, non-impact printing material, colour filters, cosmetics or toners comprising high-molecular-weight organic material and a compound of formula (1), in a colour-producing amount.

In a special embodiment of the process according to the invention, toners, paints, inks or coloured plastics are produced by processing master batches of toners, paints, inks or coloured plastics in roll mills or mixing or grinding apparatus.

A colour-producing amount of the compound of formula (1) means in the present invention normally from 0.0001 to 99.99% by weight, preferably from 0.001 to 50% by weight and especially from 0.01 to 50% by weight, based on the total weight of the material coloured or pigmented therewith.

The coloured/pigmented high-molecular-weight materials obtained, such as, for example, plastics, fibres, paints and prints, are distinguished by very high colour intensity, high saturation, good fastness to overspraying, good migration-stability, good fastness to heat, light and weathering and by a high gloss and good IR reflectance behaviour.

In order to improve the light fastness properties, UV absorbers are advantageously mixed into the plastics or polymeric particles to be coloured with the compound of formula (1) according to the invention. The amount of UV absorber can vary within a wide range; advantageously there is used from 0.01 to 1.0% by weight, especially from 0.05 to 0.6% by weight, more especially from 0.1 to 0.4% by weight, of a UV absorber, based on the weight of the plastics or polymeric particles.

The following Examples serve to illustrate the invention. Unless otherwise indicated, the parts are parts by weight and the percentages are percentages by weight. The temperatures are given in degrees Celsius. The relationship between parts by weight and parts by volume is the same as that between grams and cubic centimetres.

### EXAMPLE 1

### General Procedure for Oxidative Dimerization

A solution of 5-phenyl-4-carbethoxy-1,3-dihydro-pyrrol-2-one (10.0 g, 0.04 mol) is heated at 130° C., under stirring in dry DMF (dimethyl formamide) (100 ml). Slow stream of oxygen gas is bubbled through the solution and progress of the reaction is monitored by TLC (˜3 hours). After completion of the reaction, it is poured on water (5-times). Bluish suspension is filtered through Buchner funnel to give dark violet colored crude product (11.0 g). The crude product is purified by selective precipitation from hexane-ethyl acetate solvent mixture to give (2.0 g, 20%) of the pure product. ¹H NMR (dmso): δ1.1(6H, t), 4.1(4H, q), 7.4-7.7 (10H, m), 11.2 (2H, s). All other compounds given in the Table 1 (entries 1, 3, 4, 7) are obtained following the same procedure.

### EXAMPLE 2

### General Procedure for N-butylation

Dimer obtained from process of Example 1 (4.5 g, 0.009 mol) is taken in dry DMF (70 ml) under nitrogen atmosphere and the solution is cooled to 5° C. To this sodium hydride 50% (0.94 g, 0.019 mol) is added in portions. After some time n-butyl bromide (4.65 g, 0.034 mol) is added in one lot. Reaction was monitored by TLC and after the completion, it is poured on brine. Aqueous part is extracted with ethyl acetate, dried over anhydrous sodium sulfate. Evaporation of organic layer gives the crude violet colored product. It is purified on silica gel column using hexane-ethyl acetate solvent mixture (10:90) to give (3.3 g, 58%) of the pure product. ¹H NMR (dmso): δ0.6(6H, t), 0.8(10H, m), 1.2(4H, t), 3.4(4H, t), 3.9(4H, q), 7.7 (10H, s). All other compounds given in the Table 1 (entries 2, 5, 6) are obtained following the same procedure.

In the following Tables Et means —CH₂CH₃, Me means —CH₃, OMe means —OCH₃, t-But means —C(CH₃)₃, Ph means -Phenyl, OEt means —OCH₂CH₃ and n-But means —(CH₂)₃CH₃.

TABLE 1

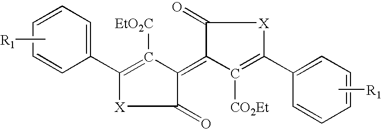

| Example | X | R₁ | Shade (PET) |
|---|---|---|---|
| 1 | N—H | H | Violet |
| 2 | N-nC₄H₉ | H | Violet |
| 3 | N—H | 4-Me | Blue |
| 4 | N—H | 4-OMe | Blue |
| 5 | N-nC₄H₉ | 4-Me | Violet |
| 6 | N-nC₄H₉ | 4-OMe | Violet |
| 7 | N—H | 4-Cl | Blue |

### EXAMPLE 8

Benzoylacrylic acid (10.0 g, 0.056 mol), cuprous chloride (2.0 g, 0.010 mol), ammonium chlorode (2.2 g, 0.041 mol) are taken in acetic anhydride (50 ml). It is gradually heated to reflux under stirring for 2 hours. After the reaction is over, it is thoroughly cooled in an ice bath. Settled solids are filtered through Buchner funnel. Solids are washed with acetic anhydride, water and ethanol to give the crude product (5.2 g). The crude solids are purified by soxhlet extraction in toluene to give pure red colored solids (3.8 g, 42%). m. p. 315° C., Anal. Calc. For C₂₀H₁₂O₄; C, 75.94; H, 3.82; Found: C, 74.79; H, 3.82. All other compounds given in the Table 2 are obtained following the above same procedure.

TABLE 2

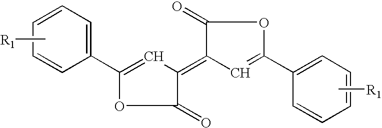

| Example | R₁ | Shade (PET) |
|---|---|---|
| 8 | H | Scarlet (fluorescent) |
| 9 | 4-Me | Red |
| 10 | 4-t-But | Red |
| 11 | 4-Ph | Red |
| 12 | 4-OMe | Red |
| 13 | 4-OEt | Red |
| 14 | 4-Cl | Red |
| 15 | 4-Br | Red |
| 16 | 3-NO₂ | Red |

### EXAMPLE 17

2.0 g of the above dimmer from Example 8 is taken in acetic acid (50 ml). Ammonia gas is then bubbled through the solution under reflux for 2 hours. Reaction mixture is cooled to 70° C. and filtered. Solids are washed with water, ethanol and ether. The crude product is purified by continuous soxhlet extraction to give (1.4 g, 70%) of violet colored product. Anal. Calc. For C₂₀H₁₄N₂O₂; C, 76.42; H, 4.49; N, 8.90; Found: C, 75.84; H, 5.10; N, 7.70. All other compounds given in the Table 3 are obtained following the above same procedure.

TABLE 3

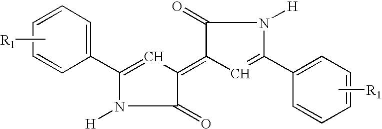

| Example | R₁ | Shade (PET) |
|---|---|---|
| 17 | H | Violet |
| 18 | 4-Me | Violet |
| 19 | 4-t-But | Violet |
| 20 | 4-Ph | Violet |
| 21 | 4-OMe | Grey |
| 22 | 4-OEt | Grey |
| 23 | 4-Cl | Red |
| 24 | 4-Br | Red |
| 25 | 3-NO₂ | Red |

### EXAMPLE 26

2,3-Bis-benzoylamino-succinic acid (5.0 g, 0.014 mol) (synthesized as given in Stachel, S. D. et al. Arch. Pharm. 312, 968, 1979) is taken in thionyl chloride (50 ml) and refluxed for 2 hours. Excess thionyl chloride is distilled off. Traces of thionyl chloride are removed by toluene co-distillation. It is cooled to room temperature and water is added to it. The crude mass is then filtered and dried in an oven till constant weight. The red colored pure product obtained, weighted (3.5 g, 78%). Anal. Calc. For C₁₈H₁₀N₂O₄; C, 67.90; H, 3.17; N, 8.8; Found: C, 67.07; H, 4.95; N, 8.81. All other compounds given in the Table 4 are obtained following the above same procedure.

TABLE 4

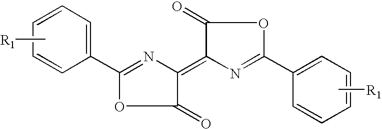

| Example | R₁ | Shade (PET) |
|---|---|---|
| 26 | H | Red |
| 27 | 4-Me | Red |
| 28 | 4-Cl | Red |
| 29 | 4-NO₂ | Red |

### EXAMPLE 30

Benzamidine free base (6.0 g, 0.049 mol) and dimethyl acetylenedicarboxylate (3.6 g, 0.025 mol) are taken in benzene (ca. 50 ml). The deep red colored solution obtained is heated to reflux for 2 hours. The cooled mixture is filtered to give (9.0 g) of the crude. The crude product is taken in minimum amount of warm DMF and poured on large excess of water under stirring. The solids separated (2.1 g) are filtered and soxhlet extracted with methanol for 16 hours. The methanol insoluble pure red colored product obtained, weighted (0.8 g, 10%). Anal. Calc. For C₁₈H₁₂N₄O₂; C, 68.37; H, 3.79; N, 17.72; Found: C, 69.33; H, 3.43; N, 17.47. Methoxy derivative from Example 31 (see Table 5) is synthesized using above same method.

TABLE 5

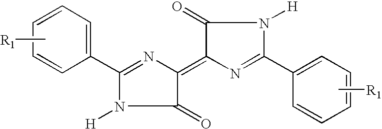

| Example | R₁ | Shade (PET) |
|---|---|---|
| 30 | H | Yellow |
| 31 | 4-OMe | Orange |

### EXAMPLE 33

5-Phenyl-4-carbethoxy-1,3-dihydro-pyrrol-2-one (2.3 g, 0.01 mol) is dissolved in acetic acid (50 ml) at 60 to 70° C. To this PTSA (0.7 g) is added, followed by addition of 2,5-dimethoxy-terephthalaldehyde (1.0 g, 0.005 mol) and temperature is increased to 100° C. Heating is continued for 3 hours and after cooling, solids are filtered through Buchner funnel. The crude product is washed with acetic acid, water, DMF and methanol. Finally the crude product is dissolved in conc. sulphuric acid, reprecipitated from water and filtered. It is then dried in an oven to give red colored pure product (1.5 g, 50%). Anal. Calc. For C₃₆H₃₂N₂O₈; C, 69.60; H, 5.15; N, 4.52; Found: C, 66.47; H, 4.98; N, 4.34. All other compounds given in the Table 6 are obtained following the above same procedure

TABLE 6

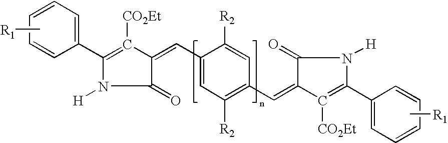

| Example | n | R₁ | R₂ | Shade (PET) |
|---|---|---|---|---|
| 32 | 0 | H | H | Red |
| 33 | 1 | H | OMe | Orange |
| 34 | 1 | H | H | Orange |
| 35 | 2 | H | H | Yellow |
| 36 | 0 | 4-Me | H | Red |
| 37 | 1 | 4-Me | H | Orange |
| 38 | 2 | 4-Me | H | Yellow |
| 39 | 0 | 4-Cl | H | Red |
| 40 | 1 | 4-Cl | H | Orange |
| 41 | 2 | 4-Cl | H | Yellow |
| 42 | 0 | 4-OMe | H | Red |
| 43 | 1 | 4-OMe | H | Orange |

### EXAMPLE 44

5-(p-Methylphenyl)-4-carbethoxy-1,3-dihydro-pyrrol-2-one (5.1 g, 0.0086 mol) is taken in dry DMF (70 ml) under nitrogen atmosphere and the solution is cooled to 5° C. To this sodium hydride 50% (1.0 g, 0.04 mol) is added in portions. After some time n-butyl bromide (6.35 g, 0.046 mol) is added to it at ones. Reaction is monitored by TLC and after the completion, it is poured on brine. Aqueous part is extracted with ethylacetate, dried over anhydrous sodium sulfate. Evaporation of organic layer gives the crude violet colored product. It is purified on silica gel column using hexane-ethyl acetate solvent mixture (10:90) to give (3.5 g,58%) of the pure product. ¹H NMR (dmso): δ0.6(6H, t), 0.8(6H, t), 1.2(4H, m), 1.4(4H, m), 2.4(6H, s), 3.4(4H, t), 4.0(4H, q), 7.2-8.3(14H, m). All other compounds given in the Table 7 are obtained following the same procedure.

TABLE 7

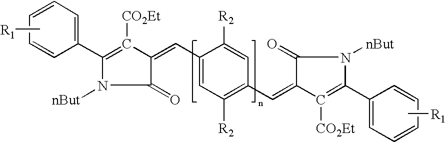

| Example | n | R₁ | R₂ | Shade (PET) |
|---|---|---|---|---|
| 44 | 1 | 4-Me | H | Gold Yellow |
| 45 | 1 | H | H | Orange |
| 46 | 2 | H | H | Yellow |
| 47 | 0 | 4-Me | H | Red |
| 48 | 0 | H | H | Red |
| 49 | 2 | 4-Me | H | Yellow |
| 50 | 0 | 4-Cl | H | Red |
| 51 | 1 | 4-Cl | H | Orange |
| 52 | 2 | 4-Cl | H | Yellow |
| 53 | 1 | 4-OMe | H | Red |

### EXAMPLE 54

Terephthalaldehyde (2.0 g, 0.015 mol), sodium acetate (7.5 g, 0.091 mol) and acetic anhydride 70 ml) are taken in round bottom flask and heated to 90° C. under nitrogen atmosphere. To this is added β-Benzoylpropionic acid (16.0 g, 0.09 mol). Temperature of the reaction is maintained for 3 hours and after cooling solids are filtered through Buchner funnel, washed with acetic acid, water and methanol. The crude product is heated in DMF at 80-90° C. for 2 hours. After filtration and washing with water, it is dried in an oven till constant weight to give (5.2 g, 83%) of the pure product. Anal. Calc. For C₂₈H₁₈O₄; C, 78.37; H, 4.34; Found: C, 78.87; H, 4.45. The compounds of Examples 55 and 56 (see table 8) are synthesized using above same method.

TABLE 8

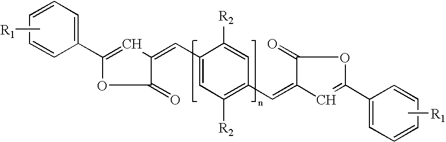

| Example | n | R₁ | R₂ | Shade (PET) |
|---|---|---|---|---|
| 54 | 1 | H | H | Orange |
| 55 | 1 | H | OMe | Orange |
| 56 | 2 | H | H | Yellow |

### EXAMPLE 57

Above prepared lactone of Example 54 (11.5 g, 0.027 mol) is taken in acetic acid (200 ml). Ammonia gas is bubbled through it and the solution is refluxed for 2 hours. After completion of the reaction, it is cooled and filtered. The solids are washed with acetic acid, water and methanol. Final purification is done by stirring the solids in DMF at room temperature for 4-5 hours. Filtered solids are then washed with water, methanol and dried in an oven till constant weight to give (10.2 g, 89%) of the orange colored pure product. Anal. Calc. For C₂₈H₂₀N₂O₂; C, 80.76; H, 4.80; N, 6.73 Found: C, 80.37; H, 4.14; N, 7.21. Example 58 (see Table 9) is synthesized using above same method.

### EXAMPLE 59

Above lactam from Example 57 (6.5 g, 0.020 mol) is taken in dry DMF (90 ml) under nitrogen atmosphere and the solution is cooled to 5° C. To this sodium hydride 50% (1.8 g, 0.075 mol) is added in portions. After some time n-butyl bromide (9.90 g, 0.075 mol) is added to it at ones. Reaction is monitored by TLC and after completion, it is poured on brine. Aqueous part is extracted with ethyl acetate, dried over anhydrous sodium sulfate. Evaporation of organic layer gives the crude orange colored product. It is purified on silica gel column using hexane-ethyl acetate solvent mixture (10:90) to give orange colored (2.0 g, 18%) of the pure product. m. p. 160° C.; ¹H NMR (cdcl₃): δ0.8(6H, t), 1.2(4H, t), 1.4(4H, m), 3.7(4H, t), 6.2(2H, s), 7.2-7.6(16H, m).

TABLE 9

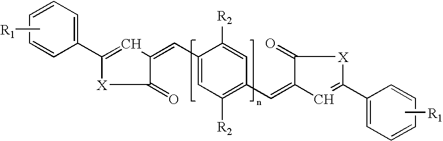

| Example | X | n | R₁ | R₂ | Shade (PET) |
|---|---|---|---|---|---|
| 57 | NH | 1 | H | H | Orange |
| 58 | NH | 2 | H | H | Yellow |
| 59 | N-nBut | 1 | H | H | Orange |

### EXAMPLE 60

Terephthalaldehyde (7.0 g, 0.055 mol), sodium acetate (14 g, 0.017 mol) and acetic anhydride (150 ml) are taken in round bottom flask and heated to 90° C. under nitrogen atmosphere. To this is added hippuric acid (30.0 g, 0.167 mol). Temperature of the reaction is maintained for 4 hours and after cooling yellow solids are filtered through Buchner funnel, washed with acetic acid, water and methanol. The crude product is heated in DMF at 80-90° C. for 2 hours. After filtration and washing with water, it is dried in an oven till constant weight to give (14.5 g, 66%) of the pure product. Anal. Calc. For C₂₆H₁₆N₂O₄; C, 74.28; H, 3.84;N, 6.66 Found: C, 73.57; H, 3.67; N, 6.79.

TABLE 10

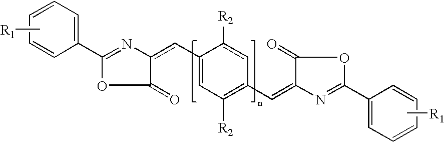

| Example | n | R₁ | R₂ | Shade (PET) |
|---|---|---|---|---|
| 60 | 1 | H | H | Yellow (fluorescent) |
| 61 | 0 | H | H | Orange |
| 62 | 2 | H | H | Yellow |

### EXAMPLE 63

Above oxazoline based compound of Example 61 (1.0 g, 0.0023 mole) is taken in acetic acid (30 ml). The reaction mixture is refluxed while ammonia gas is bubbled through the solution for 2 hours. The reaction mixture is filtered after cooling and washed with acetic acid, water and methanol. The crude mass is stirred in DMF at room temperature for 15 hours followed by boiling in water for 3 hours. Final wash is given with methanol and dried in an oven till constant weight. The pure product obtained in this way gives (2.5 g, 22%) yield of the pure product. Anal. Calc. For C₂₆H₁₈N₄O₂; C, 74.64; H, 4.34;N, 13.79 Found: C, 72.75; H, 4.19; N, 13.60.

TABLE 11

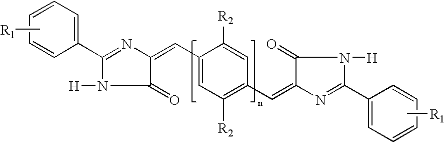

| Example | n | R₁ | R₂ | Shade (PET) |
|---|---|---|---|---|
| 63 | 1 | H | H | Orange |
| 64 | 2 | H | H | Yellow |

### DYEING EXAMPLE 1

1200.00 g of polyester granules (PET Arnite D04-300, DSM) are pre-dried for 4 hours at 130° C. and then mixed homogeneously with
2.6 g of the compound of formula 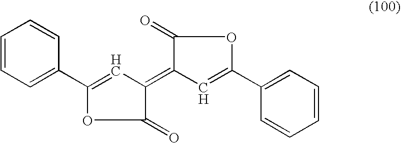
in a “roller rack” mixing apparatus for 15 minutes at 60 revolutions per minute. The homogeneous mixture is extruded in an extruder (twin screw 25 mm from Collin, D-85560 Ebersberg) with 6 heating zones at a maximum temperature of 275° C., cooled with water, granulated in a granulator (Turb Etuve TE 25 from MAPAG AG, CH-3001 Bern) and then dried for 4 hours at 130° C.

The resulting greenish yellow-coloured polyester granules have good allround fastness properties, especially good light fastness and high-temperature light fastness properties.

### DYEING EXAMPLE 2

1200.00 g of polyamide-6 granules (Ultramid B3K, BASF) are pre-dried for 4 hours at 75° C. and then mixed homogeneously with
3.5 g of the compound of formula 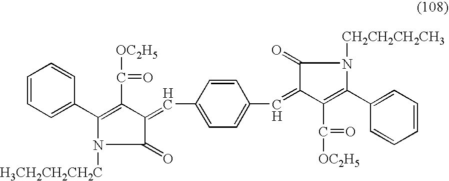
in a “roller rack” mixing apparatus for 15 minutes at 60 revolutions per minute. The homogeneous mixture is extruded in an extruder (twin screw 25 mm from Collin, D-85560 Ebersberg) with 6 heating zones at a maximum temperature of 220° C., cooled with water, granulated in a granulator (Turb Etuve TE 25 from MAPAG AG, CH-3001 Bern) and then dried for 4 hours at 75° C.

The resulting orange-coloured polyamide granules have good allround fastness properties, especially good light fastness and high-temperature light fastness properties.

1. Compounds of formula (1) 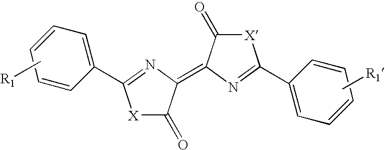
wherein
R₁ is hydrogen, hydroxy, halogen, nitro, cyano, amino, carboxy, carboxylic ester, sulfo, sulfonic ester, carboxylic amide, sulfonic amide, alkylthio, arylthio, alkoxy or aryloxy,
R′₁ is hydroxy, halogen, nitro, cyano, amino, carboxy, carboxylic ester, sulfo, sulfonic ester, carboxylic amide, sulfonic amide, alkylthio, arylthio, alkoxy or aryloxy,
X is —O—, —NH— or —N(alkyl)-, and
X′ is —O—, —NH— or —N(alkyl)-.

2. Compounds according to claim 1, wherein
R₁ is hydrogen, chloro, bromo, methyl, methoxy, ethoxy, tert.-butyl, phenyl or nitro,
R′₁ is chloro, bromo, methyl, methoxy, ethoxy, tert.-butyl, phenyl or nitro,
X is —NH—, —N(n-C₄H₉)— or —O—, and
X′ is —NH—, —N(n-C₄H₉)— or —O—.

3. Compounds according to claim 1 of formula

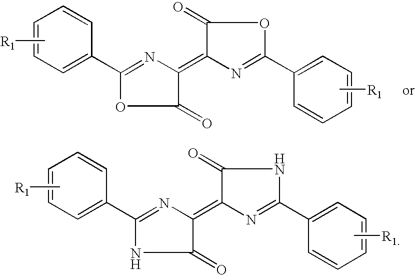

4. Compounds according to claim 2 of formula

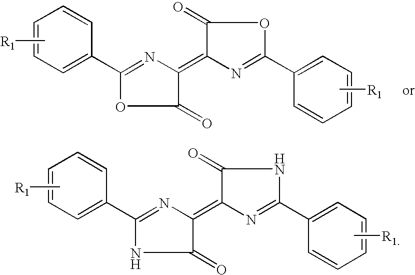

5. Process for the preparation of compounds of formula (1), which process comprises oxidation of 1 mol of compound of formula (50) and 1 mol of compound of formula (51) at elevated temperature

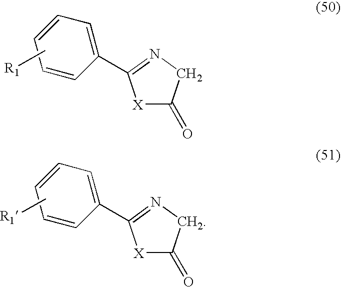

6. Coloured or pigmented plastics or polymeric coloured particles comprising compound of formula (1) according to claim 1.

7. An ink, stain, paint, roll coating, powder coating, textile, mineral oil, lubricating grease, wax, polymeric fiber, polymeric plate, polymeric moulded substrate, non-impact printing material, cosmetic, toner or optical information storage composition comprising a compound of formula (1) according to claim 1.

8. A paint or coating composition of claim 9 which is an automobile paint, high-solids coating, water-containing paint or metallic paint.

9. A colour filter, for liquid crystal display device or charge-coupled device comprising a compound of formula (1) according to claim 1.
